(19) United States
(12) Patent Application Publication
(10) Pub. No.: US 2025/0198977 A1
(43) Pub. Date: Jun. 19, 2025

(54) DETECTION METHOD, DRIED ALGAE MANUFACTURING METHOD, DRIED ALGAE, AND DRIED ALGAE QUALITY MANAGEMENT METHOD

(71) Applicant: NATIONAL INSTITUTE FOR ENVIRONMENTAL STUDIES, Ibaraki (JP)

(72) Inventors: Masanobu KAWACHI, Tsukuba-shi (JP); Shuhei OTA, Tsukuba-shi (JP); Hiroshi KOSHIKAWA, Tsukuba-shi (JP)

(21) Appl. No.: 18/848,474

(22) PCT Filed: Mar. 23, 2023
(86) PCT No.: PCT/JP2023/011478

§ 371 Date: Sep. 18, 2024

(30) Foreign Application Priority Data
Mar. 23, 2022 (JP) 2022-047117

(51) Int. Cl. G01N 33/18; C12Q 1/02; G01N 21/64

(52) U.S. Cl. CPC G01N 33/1813; C12Q 1/025; G01N 21/6428; G01N 2021/6432; G01N 2333/405

(57) ABSTRACT

The present invention relates to a detection method for detecting the presence or absence of a test substance in a sample, the detection method including a measurement step of measuring a delayed luminescence A of a measurement object obtained by bringing dried algae into contact with the sample.

### TECHNICAL FIELD

The present invention relates to a detection method, a dried algae manufacturing method, dried algae, and a dried algae quality management method.

Priority is claimed on Japanese Patent Application No. 2022-047117, filed on Mar. 23, 2022, the content of which is incorporated herein by reference.

### BACKGROUND ART

In recent years, development and survey of seabed mineral resources distributed around Japan have been progressing. For example, non-ferrous metal resources in seabed hydrothermal deposit areas such as Okinawa Trough and seabed mineral resources such as rare earths in the sea area around Minami-Torishima are attracting attention. It is assumed that these resources are to be useful for the stable supply of mineral resources which are depleted worldwide, and there is a high level of interest in these resources both domestically and internationally.

While research and development for commercial mining and ore lifting is promoted, in 2017, Japan Oil, Gas and Metals National Corporation (JOGMEC) succeeds in a pilot test of ore lifting, in which ore collected by drilling the seabed at a depth of approximately 1,600 m is continuously raised to the sea surface using a submersible pump, for the first time in the world.

In a case of full-scale resource development, various operations are performed, such as installation and operation of drilling machines and riser pipes in the deep sea and offshore, offshore treatment of raised ore water, and loading from a mining mother ship to a transport ship. In any of the work processes, it is necessary to take measures considering a risk of accidents and an associated risk of mineral leakage, and there is a demand for a bioassay technology that can be easily implemented in various situations, including onboard laboratories.

In the related art, the presence of a chemical substance has been estimated by exposing algae to the chemical substance and using the growth of the algae or the amount of delayed luminescence as an index.

As a bioassay method using the living cells of algae, OECD Test No. 201 is known. In addition, a method using delayed luminescence (Patent Document 1) or short-time methods using delayed luminescence [Non Patent Documents 1 to 2, and ISO registration (ISO 23734:2021)] have been proposed.

### CITATION LIST

### Patent Document

### [Patent Document 1]

PCT International Publication No. WO2011/055658

### Non-Patent Documents

[Non-Patent Document 1] “Rapid ecotoxicological bioassay using delayed fluorescence in the green alga Pseudokirchneriella subcapitata”, M. Katsumata, T. Koike, M. Nishikawa, K. Kazumura, H. Tsuchiya, WATER RESEARCH (2006) 40:3393-3400.

### [Non-Patent Document 2]

“Utility of Delayed Fluorescence as Endpoint for Rapid Estimation of Effect Concentration on the Green Alga Pseudokirchneriella subcapitata”, M. Katsumata, T. Koike, K. Kazumura, A. Takeuchi, Y. Sugaya, Bull. Environ. Contam. Toxicol. (2009) 83:484-487.

### SUMMARY OF INVENTION

### Technical Problem

However, in bioassays using algae in the related art, it is assumed that living cells of a subcultured strain or a frozen stock strain, which are revived as a test strain, are used. In a case of using a subcultured strain or a frozen strain, continuous subculturing, regenerating the test strain according to the test schedule, and culture steps such as preculture are required. In addition, in order to enhance reproducibility, it is necessary to control a culture environment during the test (for example, cell count, temperature conditions, light conditions, and the like). Furthermore, in assays using live algae, a standard growth inhibition test (OECD TG201) requires 72 hours, and even a short-time method using the delayed luminescence method (Non-Patent Documents 1 and 2) requires 24 hours. Therefore, it cannot be said that needs of the field for rapid evaluation results are met.

The present invention has been made to solve the above-described problems, and an object of the present invention is to provide a detection method that enables a simple and rapid assay for a test substance.

### Solution to Problem

As a result of intensive studies to solve the above-described problems, the present inventors have found that, by using dried algae, it is possible to perform a simple and rapid assay without requiring a culture step during a test, thereby achieving significant labor savings and shortening a test time, and have thus completed the present invention.

That is, the present invention has the following aspects.

(1) A detection method for detecting a presence or absence of a test substance in a sample, the detection method including:
a measurement step of measuring a delayed luminescence A of a measurement object obtained by bringing dried algae into contact with the sample.

(2) The detection method according to (1),
in which, in the measurement step, the delayed luminescence A of the measurement object is measured by bringing the dried algae into contact with water and the sample, or by bringing the dried algae into contact with the sample containing water.

(3) The detection method according to (2),
in which, in the measurement step, a value of the delayed luminescence A is measured within 90 minutes from a point of time at which the dried algae are brought into contact with the water.

(4) The detection method according to any one of (1) to (3),
in which the presence or absence of the test substance in the sample is determined based on a comparison result between a value of a delayed luminescence B of a control measurement object and a value of the delayed luminescence A.

(5) The detection method according to any one of (1) to (4),
in which the test substance contains an electron transfer inhibitor which inhibits an electron transfer reaction in a photosynthesis process.

(6) The detection method according to any one of (1) to (5),
in which the test substance contains a heavy metal.

(7) The detection method according to any one of (1) to (6), further including:
a drying step of drying algae to obtain the dried algae.

(8) The detection method according to (7),
in which a drying method is an L-drying method.

(9) The detection method according to any one of (1) to (8),
in which the dried algae are on a filter.

(10) The detection method according to any one of (1) to (9),
in which algae of the dried algae include one or more kinds of algae selected from the group consisting of algae belonging to Raphidocelis genus, algae belonging to Synechococcus genus, algae belonging to Cyanobium genus, algae belonging to Prochlorococcus genus, algae belonging to Chlorella genus, and algae belonging to Parachlorella genus.

(11) The detection method according to any one of (1) to (10),
in which algae of the dried algae include one or more kinds of algae selected from the group consisting of Raphidocelis subcapitata NIES-35, Synechococcus sp. NIES-969, Cyanobium sp. NIES-981, and Prochlorococcus sp. NIES-2885.

(12) The detection method according to any one of (1) to (11),
in which the measurement step includes a measurement step of measuring a delayed luminescence A of a measurement object by bringing an L-dried matter of algae, which is dried algae obtained by an L-drying method, into contact with the sample.

(13) The detection method according to any one of (1) to (12),
in which the measurement step includes a measurement step of measuring a delayed luminescence A of a measurement object obtained by directly bringing the dried algae into contact with the sample containing water.

(14) A dried algae manufacturing method used in the detection method according to any one of (1) to (13), the manufacturing method including:
a drying step of drying algae to obtain the dried algae.

(15) The manufacturing method according to (14),
in which a drying method is an L-drying method.

(16) The manufacturing method according to (15),
in which, in the L-drying method, the algae in a protective medium are dried, and
the protective medium contains sugars and a Tris buffer.

(17) The manufacturing method according to any one of (14) to (16),
in which the algae are dried on a filter.

(18) The manufacturing method according to any one of (14) to (17),
in which, after the drying step, the dried algae are stored under atmospheric pressure.

(19) The manufacturing method according to any one of (14) to (18),
in which the algae include one or more kinds of algae selected from the group consisting of algae belonging to Raphidocelis genus, algae belonging to Synechococcus genus, algae belonging to Cyanobium genus, algae belonging to Prochlorococcus genus, algae belonging to Chlorella genus, and algae belonging to Parachlorella genus.

(20) The manufacturing method according to any one of (14) to (19),
in which the algae include one or more kinds of algae selected from the group consisting of Raphidocelis subcapitata NIES-35, Synechococcus sp. NIES-969, Cyanobium sp. NIES-981, and Prochlorococcus sp. NIES-2885.

(21) Dried algae used in the detection method according to any one of (1) to (13).

(22) The dried algae according to (21),
in which algae of the dried algae are placed on a filter.

(23) The dried algae according to (21) or (22),
in which algae of the dried algae include one or more kinds of algae selected from the group consisting of algae belonging to Raphidocelis genus, algae belonging to Synechococcus genus, algae belonging to Cyanobium genus, algae belonging to Prochlorococcus genus, algae belonging to Chlorella genus, and algae belonging to Parachlorella genus.

(24) The dried algae according to any one of (21) to (23),
in which algae of the dried algae include one or more kinds of algae selected from the group consisting of Raphidocelis subcapitata NIES-35, Synechococcus sp. NIES-969, Cyanobium sp. NIES-981, and Prochlorococcus sp. NIES-2885.

(25) A dried algae quality management method which is a quality management method of the dried algae used in the detection method according to any one of (1) to (13), the quality management method including:
a measurement step of bringing the dried algae into contact with an electron transfer inhibitor which inhibits electron transfer in a photosynthesis process to measure a delayed luminescence A′; and
an evaluation step of evaluating quality of the dried algae based on a comparison result between a value of a delayed luminescence B′ of a control measurement object and a value of the delayed luminescence A′.

(26) The quality management method according to (25),
in which the electron transfer inhibitor is one or more selected from the group consisting of 3-(3,4-dichlorophenyl)-1,1-dimethylurea (DCMU), hydroxylamine (HA), and sodium azide.

### Advantageous Effects of Invention

According to the present invention, it is possible to provide a detection method that enables a simple and rapid assay for a test substance, a dried algae manufacturing method used in the method, dried algae, and a dried algae quality management method.

### BRIEF DESCRIPTION OF DRAWINGS

FIG. 1 is a flowchart showing one embodiment of the method according to the present invention.

FIG. 2 is a schematic diagram showing an example of the detection method according to one embodiment of the present invention.

FIG. 3 is a schematic diagram showing an example of the detection method according to one embodiment of the present invention.

FIG. 4 is a schematic diagram showing an example of the detection method according to one embodiment of the present invention.

FIG. 5 is a graph schematically showing an example of a temporal change in value of a delayed luminescence A and value of a control delayed luminescence B in the detection method according to one embodiment of the present invention, as well as a difference in value between the delayed luminescence A and the delayed luminescence B.

FIG. 6 is a graph showing test results of detection tests for various chemical substances using an L-dried matter in Examples.

FIG. 7 is a schematic diagram showing an electron donor and an electron acceptor in a photosynthetic electron transfer reaction and delayed luminescence, as well as changes in oxidation-reduction potentials thereof.

FIG. 8 is an image showing an L-dried matter on a filter obtained in Examples.

FIG. 9 is a graph showing test results of a detection test for DCMU using the L-dried matter on a filter in Examples.

FIG. 10 is a graph showing test results of a detection test for ammonia nitrogen using the L-dried matter on a filter in Examples.

FIG. 11 is an estimated result of half-maximal effective concentration (EC₅₀) of zinc in the detection test using the L-dried matter in Examples.

FIG. 12 is an estimated result of half-maximal effective concentration (EC₅₀) of a Cy metal mixed solution in the detection test using the L-dried matter in Examples.

FIG. 13 is a graph showing evaluation results of storage stability of the L-dried matter in Examples at 4° C.

FIG. 14 is a graph showing evaluation results of storage stability of the L-dried matter in Examples at 37° C.

FIG. 15 is a graph showing results of an accelerated degradation test of the L-dried matter in Examples.

FIG. 16 is a graph showing storage stability of an L-dried matter of NIES-35 in Examples at 4° C., 20° C., or 37° C.

FIG. 17 is a graph showing storage stability of an L-dried matter of NIES-981 in Examples at 4° C., 20° C., or 37° C.

FIG. 18 is a graph showing results of a quality management method for the L-dried matter using DCMU in Examples.

### DESCRIPTION OF EMBODIMENTS

Hereinafter, embodiments of the detection method, the dried algae manufacturing method, the dried algae, and the dried algae quality management method of the present invention will be described.

### <<Detection Method>>

### (Measurement Step)

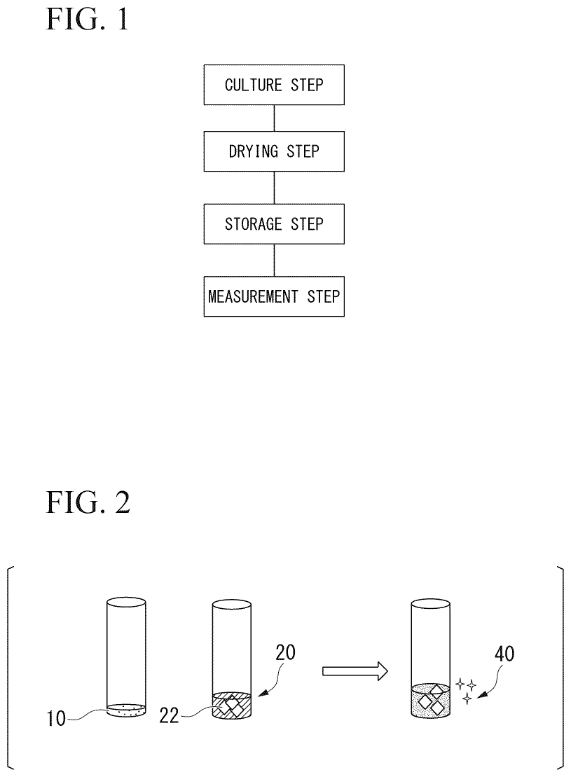

The detection method according to the embodiment is a method for detecting the presence or absence of a test substance in a sample, the detection method including a measurement step of measuring a delayed luminescence A of a measurement object obtained by bringing dried algae into contact with the sample (FIG. 1).

FIG. 2 is a schematic diagram showing an example of the detection method according to one embodiment of the present invention. In the example shown in FIG. 2, dried algae 10 and a sample 20 are mixed to bring the dried algae 10 into contact with the sample 20, and delayed luminescence emitted from a measurement object 40 thus obtained is measured.

The delayed luminescence is also referred to as delayed fluorescence (DF). The delayed luminescence is a phenomenon in which surplus energy, not utilized during a process of transferring light energy to a photochemical system II and a photochemical system I by electron in photosynthesis, is re-emitted as light energy primarily at reaction center of the photochemical system II. The delayed luminescence can also be understood as a reverse reaction of the photosynthetic reaction.

The delayed luminescence is a phenomenon observed in plant cells as a normal physiological reaction, but the delayed luminescence can be altered by substances which inhibit the photosynthetic reaction. These changes in delayed luminescence can be used to detect the substance as a test substance.

Here, the “inhibition” of the photosynthetic reaction includes both direct inhibition and indirect inhibition of the photosynthetic reaction. In addition, the “inhibition” encompasses both complete inhibition and partial inhibition.

In the method according to the present embodiment, it is not necessary to specify the type of the test substance to be detected. In the case where the test substance is detected, the type of the test substance can be specified by using a known analysis method such as mass spectrometry in combination.

In the detection method, “detection” includes cases in which the presence of the test substance in the sample is identified or estimated as a result of measuring the delayed luminescence.

In the detection method according to the present embodiment using the delayed luminescence, it is possible to detect the test substance in an amount which inhibits the photosynthetic reaction either directly or indirectly.

Substances which directly or indirectly affect the delayed luminescence and their quantities are of great significance due to concerns about environmental impact, and the ability to detect these substances is highly valuable.

As the substance which inhibits the photosynthetic reaction, electron transfer inhibitors described later, heavy metals, ammonia nitrogen, and pesticides are exemplary examples.

The above-described sample is not particularly limited, and seawater, freshwater, river water, lake water, rainwater, groundwater, drainage, factory drainage, steel mill drainage, agricultural wastewater, effluent, factory wastewater, soil, sewage, sludge, incineration ash, mine drainage, and treated products thereof are exemplary examples. As the treated product, products which have undergone heat treatment, extraction treatment, concentration treatment, filtration, centrifugation, or the like are exemplary examples.

These samples can be collected from the environment. In a case of detecting marine elution of heavy metals accompanying the raising of seabed ore, the above-described seawater is preferably obtained from a mining device, an ore raising device, or a vicinity of the ore bed.

A conventional assay using the delayed luminescence with an algal culture solution containing living algal cells is a known method in the related art. However, since the conventional assay is an evaluation method which utilizes cell proliferation of the living cells, it takes time to obtain results.

On the other hand, in the detection method according to the present embodiment, by using dried algae instead of the algal culture solution, the dried algae can be used like a powder reagent or test paper, enabling a simple and rapid assay, resulting in significant labor savings and a reduction in assay time.

Furthermore, the present inventors have found that, by using the dried algae, delayed luminescence at a very high value (referred to as “short-term life delayed luminescence”) is observed immediately after addition of water to the dried algae (hereinafter referred to as “rehydration”). This is a phenomenon not observed in the conventional bioassay using living cells, and is a specific phenomenon observed after the dried algae are rehydrated.

In this way, by using the dried algae and utilizing the short-term life delayed luminescence, the delayed luminescence is generated with an extremely high value immediately after the rehydration, and a difference from the control is amplified, enabling a short-time and high-precision test.

Details of the occurrence of the short-term life delayed luminescence are not clear, but the following mechanism is considered.

In a normal photosynthetic reaction, it is considered that, after charge separation between an electron donor and an electron acceptor, electron transfer proceeds. In a case where the electron transfer is hindered during the process, charge recombination occurs, resulting in delayed luminescence.

On the other hand, in the dried algae, it is considered that the electron donor and the electron acceptor are in a state which the charges are separated in advance, and it is considered that the short-term life delayed luminescence is generated by charge recombination due to the rehydration of the dried algae.

From such a viewpoint, as the example of the detection method according to the embodiment, it is preferable to be a method for detecting the presence or absence of a test substance in a sample, the method including a measurement step in which the delayed luminescence A of the measurement object is measured by bringing the dried algae into contact with water and the above-described sample, or by bringing the dried algae into contact with the above-described sample containing water.

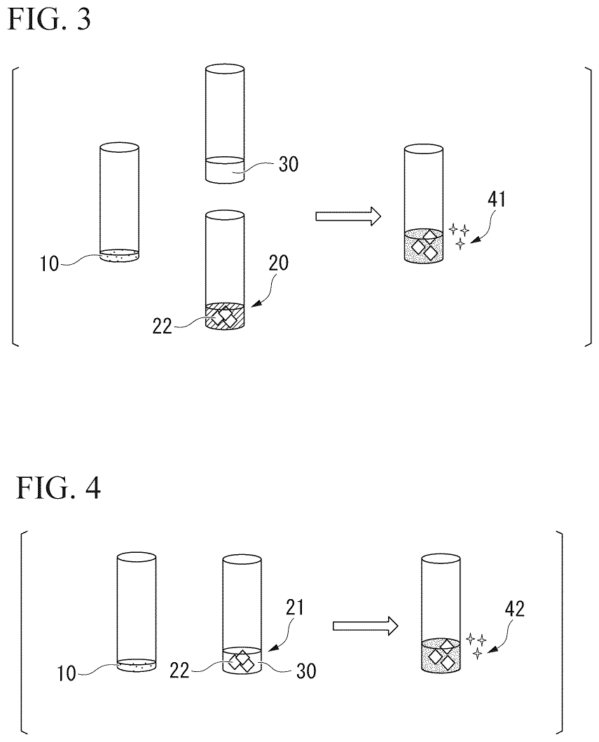

FIGS. 3 and 4 are schematic diagrams showing examples of the detection method according to other embodiments of the present invention.

In the example shown in FIG. 3, dried algae 10, water 30, and a sample 20 are mixed to bring the dried algae 10 into contact with the sample 20, and delayed luminescence emitted from a measurement object 41 thus obtained is measured.

In the example shown in FIG. 4, a sample 21 contains water; and dried algae 10 and the sample 21 containing water are mixed to bring the dried algae 10 into contact with the sample 21, and delayed luminescence emitted from a measurement object 42 thus obtained is measured.

As the sample containing water, for example, seawater and river water as mentioned above may be used, but it is not limited to these.

Here, the contact between the dried algae and the water includes operation of adding water to the dried algae.

An amount of water to be added to the dried algae in the above-described measurement object may be, for example, based on mass, in a ratio of dried algae:water=1:1 or more, which may be 1:1 to 1:1000 or 1:1 to 1:100.

For example, 0.1 to 10 mL, preferably 3 to 4 mL of water, is added to 100 μL of a dried cell suspension (concentrated liquid) to rehydrate the dried algae. The number of algal cells contained in the 100 μL of the cell suspension varies depending on the type of algae and cell morphology, and the cell suspension can contain, for example, 1×10⁸ cells to 3×10⁹ cells of algae.

The short-term life delayed luminescence is observed immediately after the rehydration, often reaching its peak light intensity around 30 minutes and maintaining a high level for up to approximately 90 minutes.

From such a viewpoint, in the above-described measurement step, it is preferable that the value of the delayed luminescence A is measured within 90 minutes from a point of time at which the dried algae are brought into contact with the water, and from the viewpoint that a coefficient of variation can be further reduced, it is more preferable to measure the value of the delayed luminescence A within 60 minutes and it is still more preferable to measure the value of the delayed luminescence A within 40 minutes.

Since the delayed luminescence is observed immediately after the rehydration, the measurement start time after the rehydration is not particularly limited. For example, the value of the delayed luminescence A may be measured immediately after the rehydration (0 seconds after rehydration) up to 90 minutes, or the value of the delayed luminescence A may be measured 10 seconds after the rehydration up to 60 minutes.

As described above, since the delayed luminescence is a phenomenon universally observed in algae with photosynthetic ability, the presence or absence of the test substance can be detected with higher accuracy by comparing the value of the delayed luminescence with a value of control delayed luminescence.

It is preferable that the detection method according to the embodiment is a method for detecting the presence or absence of the test substance in the sample, the detection method including a measurement step of measuring the delayed luminescence A by bringing the dried algae into contact with the sample, in which the presence or absence of the test substance in the sample is determined based on a comparison result between a value of a delayed luminescence B of a control measurement object and the value of the delayed luminescence A.

As the delayed luminescence B of the control measurement object, a delayed luminescence B of a measurement object obtained by not bringing the dried algae into contact with the above-described sample or obtained by bringing the dried algae into contact with a control sample containing the above-described test substance with a known amount is an exemplary example.

As the case of bringing into contact with the control sample containing the above-described test substance with a known amount, in FIG. 2 described above, mixing of a control sample containing a test substance 22 with a known amount, instead of the sample 20, is an exemplary example.

The control sample may not contain the test substance; in other words, the amount of the test substance with a known amount may be 0.

As the case of not bringing the dried algae into contact with the above-described sample, in FIG. 3 described above, mixing of the dried algae 10 and the water 30, instead of mixing of the dried algae 10, the water 30, and the sample 20, is an exemplary example.

In a case where there is a difference between the value of the delayed luminescence B and the value of the delayed luminescence A as the comparison result, it can be determined that the test substance is present in the sample.

In a case where the value of the delayed luminescence B is not different from the value of the delayed luminescence A, it can be determined that the test substance is absent.

As the values of the delayed luminescence A and the delayed luminescence B, an amount of luminescence can be used. As the amount of luminescence, a delayed fluorescence integral (DFI), which is the sum of the amount of delayed luminescence within a predetermined time, can be adopted.

As the amount of luminescence, a value divided by a value reflecting the amount of algae in the above-described measurement object (for example, absorbance at a wavelength of 600 nm) can be adopted (for example, DFI/OD₆₀₀).

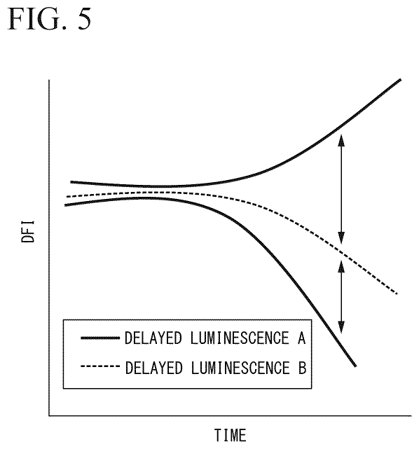

FIG. 5 is a graph showing an example of a temporal change in value of the delayed luminescence A and value of the delayed luminescence B, and a difference in value between the delayed luminescence A and the delayed luminescence B. The value of the delayed luminescence A may be higher than or lower than the value of the delayed luminescence B. In any case, it can be determined that the test substance is present in the sample.

In addition, it can be assumed that, as the difference is larger, the difference from the amount of the test substance contained in the control sample is larger. The value of the amount of the test substance contained in the sample may be estimated from the value of the amount of the test substance contained in the control sample.

The reason for the difference in whether the value of the delayed luminescence A is higher or lower compared to the value of the control delayed luminescence B is not clear, but it is considered to reflect a difference in reaction inhibiting site in the photosynthetic reaction of the chemical substance, such as whether or not the reaction center is inhibited.

In addition, since the value of the delayed luminescence changes over time from the rehydration, it is preferable to compare the value of the delayed luminescence B and the value of the delayed luminescence A at the point of time at which the same amount of time has elapsed since the sample is brought into contact or since rehydration. In a case where the above-described elapsed time differs at the acquisition point of each value between the value of the delayed luminescence B as a comparison target and the value of the delayed luminescence A, the difference is preferably within 10 minutes, more preferably within 5 minutes, and still more preferably within 3 minutes.

The measurement of the delayed luminescence B may be performed for each measurement of the delayed luminescence A, or a value of the delayed luminescence B, acquired in advance, may be used.

It is preferable that the difference in values between the delayed luminescence A and the delayed luminescence B is statistically significant. The significant difference can be evaluated based on a p-value obtained from a hypothesis test such as a t-test. For example, a P<0.05 can be considered as indicating the significant difference.

In the measurement of the delayed luminescence, it is preferable to standardize the measurement conditions of the delayed luminescence A and the delayed luminescence B in order to improve reproducibility of the values of the delayed luminescence.

From the viewpoint of equalizing an excitation state of the measurement object, it is preferable to bring the dried algae into contact with the sample, place the measurement object in a dark place, irradiate the measurement object with excitation light, and measure the delayed luminescence.

For detecting the delayed luminescence, a photodetection device equipped with a photomultiplier tube can be used. For example, a weak luminescence measurement device manufactured by Hamamatsu Photonics K.K. can be used. It is preferable that the photodetection device includes a light source for irradiating the excitation light.

As a preferred test substance in the embodiment, it is preferable that the test substance contains an electron transfer inhibitor which inhibits an electron transfer reaction in a photosynthesis process. As the electron transfer inhibitor, 3-(3,4-dichlorophenyl)-1,1-dimethylurea (DCMU), hydroxylamine (HA), sodium azide, and the like are exemplary examples. These substances can directly or indirectly inhibit the electron transfer reaction in the photosynthesis process.

It is preferable that the electron transfer inhibitor directly inhibits the electron transfer reaction in the photosynthesis process. Originally, since the delayed luminescence is likely to be generated in the photochemical system II, it is more preferable that the electron transfer inhibitor directly inhibits the electron transfer reaction in the photochemical system II.

In addition, the test substance may contain a heavy metal. In the present specification, the “heavy metal” refers to V, Cr, Mn, Fe, Co, Ni, Cu, Zn, Ga, As, Se, Rb, Sr, Y, Zr, Nb, Mo, Ru, Rh, Pd, Ag, Cd, In, Sn, Sb, Te, Cs, Ba, Hf, Ta, W, Re, Os, Ir, Pt, Au, Hg, Yl, Pb, Bi, and Po, and ions thereof. These substances inhibit the photosynthetic reaction either directly or indirectly.

Among the above-described metals, the test substance preferably contains at least one selected from the group consisting of As, Cu, Zn, Pb, and ions thereof.

The dried algae can be obtained by drying algae. As an example of a moisture content of the dried algae, a proportion of the moisture content with respect to 100% by mass of the total mass of the dried algae may be 20% by mass or less, 15% by mass or less, or 10% by mass or less. As a measuring method of the moisture content, a drying loss method can be an exemplary example.

Similarly, as an example of the moisture content of the dried algae, the moisture content with respect to 100 parts by mass of the algae contained in the dried algae may be 20 parts by mass or less, 15 parts by mass or less, or 10 parts by mass or less.

The dried algae contains algae in a dried state, and a content of the algae with respect to 100% by mass of the total mass of the dried algae may be 50% by mass or more, 70% by mass or more, 80% by mass or more, or 90% by mass or more.

The “algae contained in the dried algae” refers to a concept that includes parts of the algae, such as debris or fragments of algae cells, as long as the algae can emit the delayed luminescence.

The algae in the dried algae are not particularly limited as long as they are capable of photosynthesis and can exhibit the delayed luminescence after drying, and green algae, blue-green algae, red algae, diatoms, and dinoflagellates are exemplary examples.

From the viewpoint that the algae are widely used and easy to handle even in bioassays in the related art, the above-described algae are preferably microalgae.

As the algae of the dried algae described above, from the viewpoint of easy cultivation, distribution in the environment, and easy availability in culture collections at home and abroad, it is preferable to include one or more kinds of algae selected from the group consisting of algae belonging to Raphidocelis genus, algae belonging to Synechococcus genus, algae belonging to Cyanobium genus, algae belonging to Prochlorococcus genus, algae belonging to Chlorella genus, and algae belonging to Parachlorella genus; and it is more preferable to include one or more kinds of algae selected from the group consisting of algae belonging to Raphidocelis genus, algae belonging to Synechococcus genus, algae belonging to Cyanobium genus, and algae belonging to Prochlorococcus genus.

As the algae of the dried algae described above, it is still more preferable to include one or more kinds of algae selected from the group consisting of Raphidocelis subcapitata (Japanese name: Muremikazukimo) NIES-35 (Raphidocelis subcapitata NIES-35), Synechococcus sp. NIES-969, Cyanobium sp. NIES-981, Prochlorococcus sp. NIES-2885, Chlorella sorokiniana NIES-2169, and Parachlorella kessleri NIES-2152; and it is particularly preferable to include one or more kinds of algae selected from the group consisting of Raphidocelis subcapitata (Japanese name: Muremikazukimo) NIES-35 (Raphidocelis subcapitata NIES-35), Synechococcus sp. NIES-969, Cyanobium sp. NIES-981, and Prochlorococcus sp. NIES-2885.

These algae may be collected from the environment, and the specific strains described above can be used by utilizing strains maintained in a microbial strain preservation facility such as the National Institute for Environmental Studies (NIES).

The combination of the kind of the sample and the kind of the algae is not particularly limited.

For example, in a case where the seawater is used as the sample, the algae of the dried algae can be either marine algae or freshwater algae.

For example, in a case where the freshwater is used as the sample, the algae of the dried algae can be either marine algae or freshwater algae.

In a case where the test substance contains a heavy metal, it is preferable that the dried algae contains Raphidocelis subcapitata NIES-35 and the test substance contains at least one selected from the group consisting of As, Cu, Zn, Pb, and ions thereof.

It is preferable that the algae of the dried algae described above includes Synechococcus sp. NIES-969 and the test substance contains at least one selected from the group consisting of As, Cu, Zn, Pb, and ions thereof.

It is preferable that the algae of the dried algae described above includes Cyanobium sp. NIES-981 and the test substance contains at least one selected from the group consisting of Cu, Zn, Pb, and ions thereof.

It is preferable that the algae of the dried algae described above includes Prochlorococcus sp. NIES-2885 and the test substance contains at least one selected from the group consisting of As, Cu, Zn, Pb, and ions thereof.

For obtaining the above-described dried algae, the detection method according to the embodiment can further include, prior to the above-described measurement step, a culture step of culturing the algae and a drying step of drying the algae cultured in the culture step to obtain the above-described dried algae. In addition, the detection method according to the embodiment can further include a storage step of storing the dried algae obtained in the drying step described above, as necessary (FIG. 1).

### (Culture Step)

In the culture step, the algae, which are a raw material for the above-described dried algae, can be cultured.

As the algae to be cultured, those described as the algae of the dried algae described above are exemplary examples.

Culture conditions can be appropriately determined according to the kind of the algae. As a culture period, approximately 3 days to 2 weeks are exemplary examples, and the culture can be continued until the number of algal cells reaches saturation.

From the viewpoint of favorably maintaining the photochemical system in the cultured algae, it is preferable that light conditions during the culture are either continuous light periods or light-dark cycle conditions. A light quantum flux density to be applied during the culture may be appropriately determined depending on the algal species, and for example, it may be 50 μmol·photons·m⁻²·s⁻¹ or more or 50 to 100 μmol·photons·m⁻²·s⁻¹.

### (Drying Step)

The drying step is a step of drying the algae cultured in the culture step described above to obtain the above-described dried algae.

The drying method is not particularly limited, and natural drying, freeze drying, or the like can be performed. Since the obtained dried algae easily exhibits favorable delayed luminescence, it is preferable to dry the algae by an L-drying method (Liquid drying method).

The L-drying method is a method of drying a drying target without freezing it during the drying process. In the L-drying method, volatile components can be evaporated under vacuum or reduced pressure. In the present specification, the dried algae obtained by the L-drying method may be simply referred to as “L-dried matter”.

As the detection method according to one embodiment, a detection method for detecting the presence or absence of a test substance in a sample, the detection method including a measurement step of measuring a delayed luminescence A of a measurement object obtained by bringing the L-dried matter of algae into contact with the sample, is mentioned.

The basic principle of the L-drying method is described in a report by Sakane et al. (“Method for long-term preservation of microbial strains by L-drying method”, Microniol. Cult. Coll. December 1996, Vol. 12, p. 91-97).

A drying temperature in the drying step is preferably higher than 0° C. and 10° C. or lower, as a temperature of the drying target.

Setting conditions of a drying device can be appropriately selected, and for example, a drying device which can be set such that a trap (cooling device part) of the device is −75° C. or lower and a degree of vacuum is 20 mTorr or less can be used. For example, in a case of using a bucket-type vacuum drying container, the outside of the vacuum container can be cooled with ice or the like during the drying process.

The algae may be dried as they are using a culture medium containing the algae as the drying target. Alternatively, the algae may be dried using, as the drying target, a culture medium which has undergone dehydration treatment by centrifugation or a substitution treatment with a protective medium. It is more preferable to perform the drying using a protective medium and the above-described algae (in which the algae are suspended in the protective medium) as the drying target.

A density of the algal cells in the drying target including the protective medium and the algae can be mentioned as, for instance, 1×10⁹ to 5×10¹⁰ cells/mL. The density can be appropriately determined according to the kind of the algae used. For example, in a case of cyanobacteria, the density can be 1 to 5×10¹⁰ cells/mL, and in a case of eukaryotic algae, the density can be 1 to 5×10⁹ cells/mL.

The protective medium can contain sugars and a buffer. As the buffer, phosphate buffer and a Tris buffer are exemplary examples.

As an example of a protective medium for freshwater algae, a protective medium containing water, sodium glutamate, adonitol, sorbitol, and a phosphate buffer can be an exemplary example.

In a case where the marine algae are dried, the protective medium can contain seawater instead of the water. In addition, in this case, it is preferable to contain the Tris buffer instead of the phosphate buffer. As a result, sediment is less likely to form in the protective medium, enabling even more excellent L-drying. The seawater can be either natural seawater or artificial seawater.

As a protective medium for marine algae, a protective medium containing seawater, sodium glutamate, adonitol, sorbitol, and a Tris buffer can be an exemplary example.

According to the L-drying method, since the drying can be performed in a short time without freezing the drying target, it is considered that the photochemical system on a thylakoid membrane is more likely to be maintained in a more intact state. Therefore, it is presumed that the obtained dried algae are in a suitable state for the delayed luminescence.

The L-drying method can be performed using a dryer equipped with a vacuum pump. In addition, a commercially available L-drying apparatus can also be used. In a case where the sample to be dried is not frozen, a commercially available freeze dryer may be used.

The dried algae used in the detection method according to the embodiment may be present on a filter. In order to obtain the dried algae on a filter, the drying of the algae in the drying step described above can be performed on a filter. More specifically, the algae to be dried can be captured on a filter through filtration or the like, and the algae on the filter can be dried.

For example, in a case where a cell size of the algae is extremely small, such as a diameter of 1 μm, it may be difficult to separate the algae by centrifugal separation. In such a case, the algae cells can be concentrated using a filter, and dried.

The filter may be any filter capable of capturing at least a portion of the algae cells, and can be appropriately selected based on the size of the algae to be dried. For example, in a case where the cell size is relatively large (a diameter of more than 2 μm), use of GF/F glass fiber filter paper is an exemplary example. In a case of small cells with a diameter of less than 2 μm, a nitrocellulose filter with a pore size of approximately φ0.45 μm, known as a Millipore filter, or a cellulose acetate filter can be suitably used.

A thickness of the filter is not particularly limited, and for example, it is preferably 1 mm or less.

A cell density of the dried algal cells on the filter is not particularly limited as long as the delayed luminescence can be detected, and for example, in a case of cyanobacteria, the cell density can be 1 to 2×10⁹ cells/cm².

In a case where the algae on the filter are dried using a protective medium, for example, it is preferable to bring the algae on the filter into contact with the protective medium by either adding the protective medium dropwise to the algae on the filter or immersing the algae on the filter in the protective medium, and drying the algae in the protective medium.

After the drying using the drying device, the dried algae can be additionally dried using a desiccant such as silica gel, and the dried algae may be stored as is in the storage step.

As the detection method according to one embodiment of the present invention, there is provided a detection method for detecting the presence or absence of a test substance in a sample, the detection method including a measurement step of measuring a delayed luminescence A of a measurement object by bringing an L-dried matter of algae, which is dried algae obtained by an L-drying method, into contact with the sample, in which, in the measurement step, the delayed luminescence A of the measurement object is measured by bringing the dried algae into contact with water and the sample, or by bringing the dried algae into contact with the sample containing water, and in the measurement step, a value of the delayed luminescence A is measured within 90 minutes from a point of time at which the dried algae are brought into contact with the water.

As the detection method according to one embodiment of the present invention, there is provided a detection method for detecting the presence or absence of a test substance in a sample, the detection method including a measurement step of measuring a delayed luminescence A of a measurement object obtained by directly bringing the dried algae into contact with the sample containing water, in which, in the measurement step, a value of the delayed luminescence A is measured within 90 minutes from a point of time at which the dried algae are brought into contact with the water.

### (Storage Step)

The storage step is a step of storing the dried algae obtained in the drying step described above until it is used in the above-described measurement step.

The dried algae can be stored under atmospheric pressure. In the L-drying method in the related art, after the drying step, an enclosing operation is performed to maintain the inside of the ampoule in a vacuum state, and the ampoule is stored in this vacuum state (see Sakane et al., 1996).

However, the dried algae used in the detection method according to the embodiment can maintain potential of the favorable delayed luminescence for a long period of time even when stored under atmospheric pressure.

A storage temperature is preferably 20° C. or lower, more preferably higher than 0° C. and 20° C. or lower, still more preferably 1° C. to 10° C., and particularly preferably 4° C.

In order to achieve the temperature slightly above 0° C., low-temperature equipment such as liquid nitrogen and a deep freezer is not required, which makes storage and transportation easy.

A storage humidity is preferably low, and 50% RH or less is preferable and 30% RH or less is more preferable.

In order to avoid moisture absorption of the dried algae, the dried algae can be stored in a sealed container or a storage bag. It is preferable to accommodate a desiccant such as silica gel in the sealed container or the storage bag, together with the dried algae.

With the detection method according to the embodiment described above, by using the dried algae, a simple and rapid assay can be performed, resulting in achievement of significant labor savings and a reduction in assay time.

In the method using living cells in the related art, it is necessary to obtain a high-density pre-cultured cell which has been proliferated well.

In addition, in order to achieve reproducibility in the method using living cells in the related art, it is necessary to have technology or experience in culture operations, and there is a risk that the results may depend on the culture conditions.

On the other hand, with the detection method according to the embodiment, the operation in the measurement step is very simple, and commercially available measuring instruments can be used. This allows for the detection of the test substance with minimal variation due to the test performer.

With the detection method according to the embodiment, there are excellent advantages such as ability to quickly evaluate the detection of the test substance, ability to perform the measurement at any location, and ability to carry out the measurement step without the need for a culture facility.

As an origin of the sample, not only the marine environment but also coastal areas, rivers, lakes, and similar environments are assumed. The detection method according to the embodiment can be applied to a wide range of applications, such as drainage management for detecting the leakage of the test substance in a water environment.

### <<Dried Algae Manufacturing Method>>

The dried algae manufacturing method according to the embodiment is a dried algae manufacturing method used in the detection method according to the embodiment described above, and includes a drying step of drying algae to obtain the above-described dried algae (FIG. 1).

The dried algae manufacturing method according to the embodiment can further include a culture step of culturing the algae, prior to the above-described drying step. In addition, the dried algae manufacturing method according to the embodiment can further include a storage step of storing the dried algae obtained in the drying step described above, as necessary (FIG. 1).

Each step in the dried algae manufacturing method includes the contents described in the detection method according to the embodiment above, and a detailed description thereof will be omitted here.

The method of the drying in the drying step is preferably an L-drying method.

In the L-drying method, it is preferable to use the protective medium described above. It is preferable that the algae are dried in the protective medium, and the protective medium contains sugars and a Tris buffer.

In addition, it is preferable to dry the algae on a filter.

After the above-described drying step, the dried algae can be stored under atmospheric pressure.

The above-described algae to be dried preferably includes one or more kinds of algae selected from the group consisting of algae belonging to Raphidocelis genus, algae belonging to Synechococcus genus, algae belonging to Cyanobium genus, algae belonging to Prochlorococcus genus, algae belonging to Chlorella genus, and algae belonging to Parachlorella genus.

The above-described algae to be dried more preferably includes one or more kinds of algae selected from the group consisting of Raphidocelis subcapitata (Japanese name: Muremikazukimo) NIES-35 (Raphidocelis subcapitata NIES-35), Synechococcus sp. NIES-969, Cyanobium sp. NIES-981, Prochlorococcus sp. NIES-2885, Chlorella sorokiniana NIES-2169, and Parachlorella kessleri NIES-2152; still more preferably includes one or more kinds of algae selected from the group consisting of Raphidocelis subcapitata (Japanese name: Muremikazukimo) NIES-35 (Raphidocelis subcapitata NIES-35), Synechococcus sp. NIES-969, Cyanobium sp. NIES-981, and Prochlorococcus sp. NIES-2885.

### <<Dried Algae>>

The dried algae according to the embodiment is dried algae used in the detection method according to the embodiment described above.

The dried algae are as described in the detection method according to the embodiment above, and a detailed description thereof will be omitted here.

The dried algae are preferably placed on a filter. The “on a filter” refers to a state in which the algae are captured by the filter.

The above-described algae in the dried algae preferably includes one or more kinds of algae selected from the group consisting of algae belonging to Raphidocelis genus, algae belonging to Synechococcus genus, algae belonging to Cyanobium genus, algae belonging to Prochlorococcus genus, algae belonging to Chlorella genus, and algae belonging to Parachlorella genus.

The above-described algae in the dried algae more preferably includes one or more kinds of algae selected from the group consisting of Raphidocelis subcapitata (Japanese name: Muremikazukimo) NIES-35 (Raphidocelis subcapitata NIES-35), Synechococcus sp. NIES-969, Cyanobium sp. NIES-981, Prochlorococcus sp. NIES-2885, Chlorella sorokiniana NIES-2169, and Parachlorella kessleri NIES-2152; still more preferably includes one or more kinds of algae selected from the group consisting of Raphidocelis subcapitata (Japanese name: Muremikazukimo) NIES-35 (Raphidocelis subcapitata NIES-35), Synechococcus sp. NIES-969, Cyanobium sp. NIES-981, and Prochlorococcus sp. NIES-2885.

The dried algae can be manufactured by the dried algae manufacturing method according to the embodiment described above.

### <<Dried Algae Quality Management Method>>

The dried algae quality management method according to the embodiment is a quality management method of the dried algae used in the detection method according to the embodiment described above, the quality management method including a measurement step of bringing the dried algae into contact with an electron transfer inhibitor which inhibits electron transfer in a photosynthesis process to measure a delayed luminescence A′, and an evaluation step of evaluating quality of the dried algae based on a comparison result between a value of a control delayed luminescence B′ and a value of the delayed luminescence A′.

As the delayed luminescence B′ of the control measurement object, a delayed luminescence B′ of a measurement object obtained by not bringing the dried algae into contact with the above-described electron transfer inhibitor or obtained by bringing the dried algae into contact with a control sample containing the above-described electron transfer inhibitor with a known amount is an exemplary example.

The evaluation of quality means evaluating potential of the delayed luminescence of the dried algae. In general, in a case where the electron transfer inhibitor comes into contact with the algae, the delayed luminescence is generated, but it is assumed that the delayed luminescence is less likely to be generated in a case where components of the electron transfer system in the dried algae deteriorate during the storage process or the like.

In a case where there is a difference between the value of the delayed luminescence B′ and the value of the delayed luminescence A′ as the comparison result, it can be determined that the dried algae can exhibit favorably delayed luminescence and the quality is good.

In addition, in a case where there is no difference between the value of the delayed luminescence B′ and the value of the delayed luminescence A′, it can be determined that the dried algae cannot exhibit favorably delayed luminescence and the quality is poor.

With regard to the comparison between the value of the delayed luminescence B′ and the value of the delayed luminescence A′, the contents described in the comparison between the value of the delayed luminescence B and the value of the delayed luminescence A in the detection method according to the embodiment described above are exemplary examples. The sample can be interpreted as the electron transfer inhibitor, the delayed luminescence A′ can be interpreted as the delayed luminescence A, and the delayed luminescence B′ can be interpreted as the delayed luminescence B; and detailed descriptions thereof will be omitted here.

As the difference between the value of the delayed luminescence B′ and the value of the delayed luminescence A′ increases, the quality of the dried algae can be determined to be better.

It is preferable that the difference in values between the delayed luminescence A′ and the delayed luminescence B′ is statistically significant.

As shown in Examples described later, the dried algae according to the embodiment has a very high detection sensitivity for electron transfer inhibitor through the delayed luminescence, so that, utilizing this property, the quality of the dried algae can be evaluated with high accuracy.

As the electron transfer inhibitor, those mentioned in the detection method according to the embodiment described above are exemplary examples, and one or more selected from the group consisting of 3-3-(3,4-dichlorophenyl)-1,1-dimethylurea (DCMU), hydroxylamine (HA), and sodium azide are exemplary examples.

The electron transfer inhibitor used in the quality management method according to the embodiment is preferably 3-(3,4-dichlorophenyl)-1,1-dimethylurea (DCMU).

### Examples

Next, the present invention will be described in more detail with reference to Examples, but the present invention is not limited to Examples.

### Production of Dried Algae (1)

### (Cultivation)

20 to 30 mL of an algal strain was inoculated into 1 L of a sterilized medium for microalgae (see Tables 1 to 5), and the culture was aerated for 2 weeks under a 12:12 hour light-dark cycle. A light quantum flux density applied during the culture was set to 50 to 100 μmol·photons·m⁻²·s⁻¹. The culture temperature was maintained at 20° C. to 25° C. The tank was aerated using an aquarium air pump, with air sterilized through a syringe filter having a pore size of 0.45 μm.

The algal strain used is as follows.
Raphidocelis subcapitata NIES-35 (freshwater green algae; hereinafter referred to as “NIES-35”)
Synechococcus sp. NIES-969 (marine cyanobacteria; hereinafter referred to as “NIES-969”)
Cyanobium sp. NIES-981 (marine cyanobacteria; hereinafter referred to as “NIES-981”)
Prochlorococcus sp. NIES-2885 (marine cyanobacteria; hereinafter referred to as “NIES-2885”)

As the strain, a strain cultured for two weeks in a screw-cap test tube containing 10 mL of a medium for microalgae was used.

Formulation of an AF-6 medium (medium for freshwater algae) used for culturing the above-described NIES-35 is shown below.

TABLE 1
AF-6 medium

| | |
|---|---|
| NaNO₃ | 14 mg |
| NH₄NO₃ | 2.2 mg |
| MgSO4•7H₂O | 3 mg |
| KH₂PO₄ | 1 mg |
| K₂HPO₄ | 0.5 mg |
| CaCl₂•2H₂O | 1 mg |
| Fe-citrate | 0.2 mg |
| Citric acid | 0.2 mg |
| Biotin | 0.2 μg |
| Thiamine HCl | 1 μg |
| Vitamin B₆ | 0.1 μg |
| Vitamin B₁₂ | 0.1 μg |
| PIV metals * | 0.5 mL |
| Distilled water | 99.5 mL |

40 mg of MES buffer was added, and pH was adjusted to 6.6.

TABLE 2
* PIV metals

| | |
|---|---|
| Na₂EDTA•2H₂O | 100 mg |
| FeCl₃•6H₂O | 19.6 mg |
| MnCl₂•4H₂O | 3.6 mg |
| ZnSO₄•7H₂O | 2.2 mg |
| CoCl₂•6H₂O | 0.4 mg |
| Na₂MoO₄•2H₂O | 0.25 mg |
| Distilled water | 100 mL |

Formulation of an ESM medium (medium for marine algae) used for culturing the above-described NIES-969 and NIES-981 is shown below.

TABLE 3
ESM medium

| | |
|---|---|
| NaNO₃ | 12 mg |
| K₂HPO₄ | 0.5 mg |
| Vitamin B₁₂ | 0.1 μg |
| Biotin | 0.1 μg |
| Thiamine HCl | 10 μg |
| Fe-EDTA | 25.9 μg |
| Mn-EDTA | 33.2 μg |
| Tris(hydroxymethyl)aminomethane | 100 mg |
| Soil extract ** | |
| Seawater | 97.5 mL |

pH: 8.0

### (** Acquisition Method of Soil Extract)

200 mL of soil (preferably, soil from deciduous forest) was added to 1,000 mL of distilled water, and the mixture was heated in an autoclave at 105° C. for 1 hour, allowed to cool, and heated again in the autoclave at 105° C. for 1 hour. The supernatant was passed through a GF filter. Distilled water was added to the filtrate to adjust the volume to 1,000 mL. 10 mL of the final filtrate was dispensed into each test tube and sterilized by autoclaving at 121° C. for 20 minutes.

Formulation of a PRO-99 medium (medium for marine Prochlorococcus) used for culturing the above-described NIES-2885 is shown below.

TABLE 4
PRO-99 medium

| | |
|---|---|
| NaH₂PO₄•2H₂O | 0.779 mg |
| NH₄Cl | 4.28 mg |
| PRO-99 trace metals *** | 0.01 mL |
| Seawater | 100 mL |

TABLE 5
*** PRO-99 trace metals

| | |
|---|---|
| Na₂EDTA•2H₂O | 0.637 g |
| FeCl₃•6H₂O | 0.422 g |
| ZnSO₄•7H₂O | 2.933 mg |
| CoCl₂•6H₂O | 1.332 mg |
| MnCl₂•4H₂O | 24 mg |
| Na₂MoO₄•2H₂O | 0.839 mg |
| Na₂SeO₃ | 2.306 mg |
| NiCl₂•6H₂O | 0.37 mg |
| Distilled water | 100 mL |

### (L-Drying in Vial)

After 2 weeks, the culture solution, in which the algal cells had proliferated and reached saturation, was subjected to centrifugal separation. As centrifugal separation conditions, the mixture was dispensed into a 50 mL centrifuge tube and centrifugated at 4,400 rpm (3000×g) for 20 to 30 minutes.

After removing as much of the supernatant after the centrifugal separation as possible, 8 mL of a protective medium (Table 6) was added to the obtained pellet-like cell mass, and the cell mass was resuspended. At this time, the number of cells was measured using a hemocytometer or the like to obtain a cell suspension with a concentration of 2.4×10¹⁰ cells/ml to 3.2×10¹⁰ cells/ml.

In a case where the algae were derived from seawater, the following SM11 was used as the protective medium. In a case where the algae were derived from freshwater algae, the following SM12 was used as the protective medium.

TABLE 6
Formulation of 100 mL of protective medium

| | SM11 | SM12 |
|---|---|---|
| | (for marine) | (for freshwater) |
| Ultrapure water or distilled | 24 mL | 88 mL |
| water | | |
| Filtered seawater | 25 mL | — |
| Sugar stock solution * | 50 mL | 10 mL |
| 5M Tris buffer | 1 mL | — |
| (pH: 7.4) | | |
| 0.5M phosphate buffer | — | 2 mL |
| (pH: 7.0) | | |

* Sugar stock solution: solution obtained by dissolving 5 g of sodium glutamate, 1.5 g of adonitol, and 1 g of sorbitol in ultrapure water and making up to 100 mL in volumetric flask

The obtained cell suspension was dispensed into a cryo vial or glass vial in 100 μL portions.

The vial was placed in a metal tube rack pre-cooled to 4° C., and the metal tube rack was subjected to low-temperature vacuum drying (L-drying) for 2 to 3 hours using a low-temperature vacuum drying device (manufactured by Forest Technology Systems Ltd., FLEXI-DRY). During the drying, the lid of the cryo vial was kept open.

After the drying, the vial was taken out from the container and additionally dried in a desiccator lined with silica gel for several hours to overnight, obtaining an L-dried matter in the cryo vial. Additional drying was performed under atmospheric pressure, unlike the L-drying method in the related art.

Hereinafter, unless otherwise specified, the dried algae in Examples was the L-dried matter obtained in the cryo vial.

### Detection of Presence of Chemical Substance (1)

Various kinds of the L-dried matter obtained in the above-described cryo vial, and test water (water containing the chemical substances listed below at the concentrations (w/v) shown in Table 7) were prepared. In addition, a control (control group) which did not contain any chemical substance was also prepared.
3-(3,4-dichlorophenyl)-1,1-dimethylurea (DCMU; electron transfer inhibitor of photochemical system II)
Hydroxylamine (HA; electron transfer inhibitor of photochemical system II)
Metal mixed solution (Cy):

(metal mixed solution which was prepared by simulation based on formulation of a rock eluate from a core sample collected during a survey voyage of “Chikyu” by the Japan Agency for Marine-Earth Science and Technology; mainly containing lead and zinc (Yamagishi et al. Ecotoxicology (2018) 27:1303-1309))
Copper
Zinc
Lead
Arsenic: in NIES-35, NIES-969, and NIES-2885, test water containing As(V) at 1 ppm (w/v) was also used for examination.

In a case of tests using freshwater algae, distilled water was used as the test water or control water, and in a case of tests using marine algae, filtered and sterilized seawater was used as the test water or control water.

Using a cuvette as a measurement container, 100 μL of the above-described cell suspension was dried to obtain 1 unit of the L-dried matter, and resuspended (rehydrated) in 3.5 mL of test water or control water.

After the rehydration, the L-dried matter and the test water or control water were rapidly and thoroughly mixed by pipetting to obtain a measurement sample (measurement object).

The sample was set in a weak luminescence measurement device (manufactured by Hamamatsu Photonics K.K., weak luminescence coefficient device, Type-7100). After exposure with excitation light from a light source attached to the device, delayed luminescence was measured immediately after a point of time of the rehydration (within 30 seconds), 15 minutes later, 30 minutes later, 60 minutes later, and 90 minutes later. The measurement time for each delayed luminescence was set to 10 seconds.

The sum of amounts of delayed luminescence (photon count) measured over 10 seconds was calculated and defined as the delayed fluorescence integral (DFI). In addition, for the sample after the rehydration, an absorbance at a wavelength of 600 nm with an optical path length of 1 cm was simultaneously measured. The DFI value per cell number of the L-dried matter was obtained by dividing the DFI by the value (DFI/OD₆₀₀).

The significant difference was determined by the T-test of three independent measurements. In a case where the DFI value was significantly higher or lower than that of the control group, it was determined that the target chemical substance or heavy metal was present in the test water.

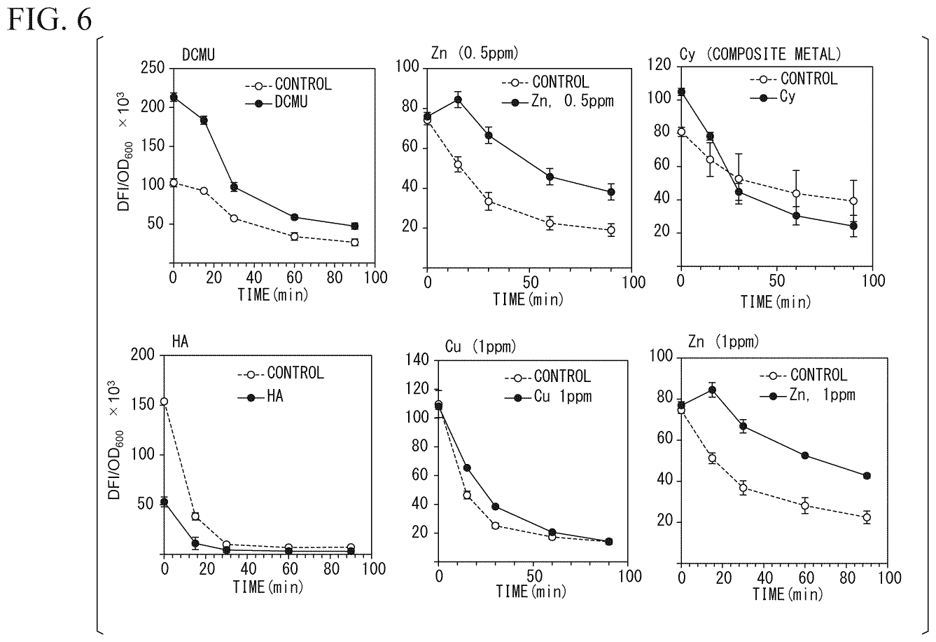

The results are shown in Table 7 and FIG. 6.

TABLE 7

| | DCMU | HA | Cy | Cu²⁺ | Zn²⁺ | Zn²⁺ | Pb²⁺ |
|---|---|---|---|---|---|---|---|
| | 1 μM | 50 μM | 1% | 1 ppm | 0.5 ppm | 1 ppm | 1 ppm |
| NIES- | 15 min ** | 15 min ** | 15 min ** | 15 min ** | 15 min ** | 15 min ** | 15 min ** |
| 35 | higher | lower | higher | lower | higher | higher | higher |
| | value | value | value | value | value | value | value |
| NIES- | 0 min ** | 0 min ** | 60 min ** | 15 min ** | 15 min ** | 60 min ** | 15 min ** |
| 969 | higher | lower | higher | lower | higher | lower | higher |
| | value | value | value → | value | value → | value | value → |
| | | | lower | | lower | | lower |
| | | | value | | value | | value |
| NIES- | 0 min ** | 0 min ** | 0 min ** | 15 min ** | 15 min ** | 15 min ** | 15 min ** |
| 981 | higher | lower | higher | higher | higher | higher | higher |
| | value | value | value | value | value | value | value |
| NIES- | 0 min ** | 0 min ** | 0 min ** | 0 min ** | 0 min ** | 30 min ** | 15 min ** |
| 2885 | lower | lower | higher | lower | higher | higher | higher |
| | value → | value | value → | value | value → | value → | value → |
| | higher | | lower | | lower | lower | lower |
| | value | | value | | value | value | value |

* P < 0.05,
** P < 0.01
upper side: shortest detection time,
lower side: comparison with control group

For each of the various chemical substances used in the test, a significantly higher and/or lower DFI value was observed compared to the control group. In addition, the time at which a significant difference was first observed after the point of time of the rehydration was recorded as the shortest detection time.

In addition, as shown in FIG. 6, very high values of delayed luminescence (short-term life delayed luminescence) were observed immediately after the rehydration.

From the above results, it was demonstrated that the assay for the target chemical substance could be performed very rapidly and with high accuracy by measuring the amount of delayed luminescence using the dried matter of algae.

Compared to the control group, it was not clear in detail whether the DFI value was higher or lower depending on the type of chemical substance. However, it is considered that the difference reflected the variation in reaction inhibiting sites of the chemical substances in the photosynthetic reaction.

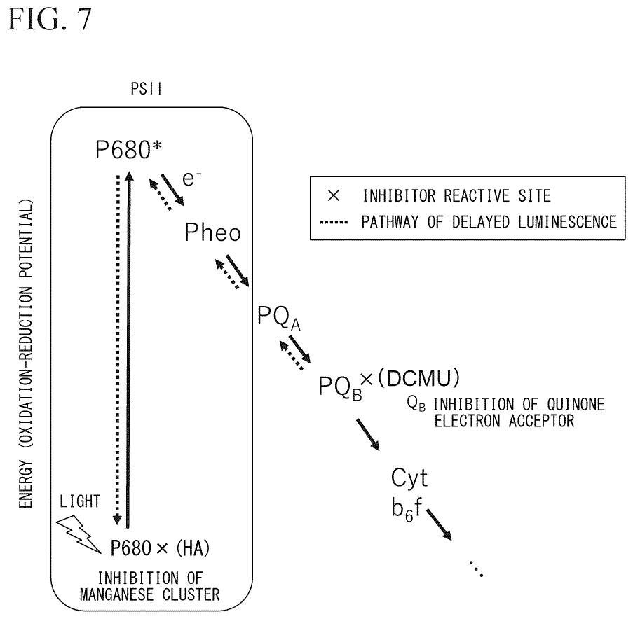

FIG. 7 is a schematic diagram showing an electron donor and an electron acceptor in the photosynthetic electron transfer reaction and the delayed luminescence, as well as changes in oxidation-reduction potentials thereof.

For example, it is known that HA inhibits function of a manganese cluster in P680. Therefore, it is considered that flow of electrons starting from P680 was hindered by the exposure to HA, and the delayed luminescence itself was also hindered.

It is known that the DCMU binds to a quinone electron-acceptor site with high specificity. Therefore, it is considered that the exposure to the DCMU hindered the electron transfer, causing electronic backflow and increasing delayed luminescence.

### Production of Dried Algae (2)

### (L-Drying on Filter)

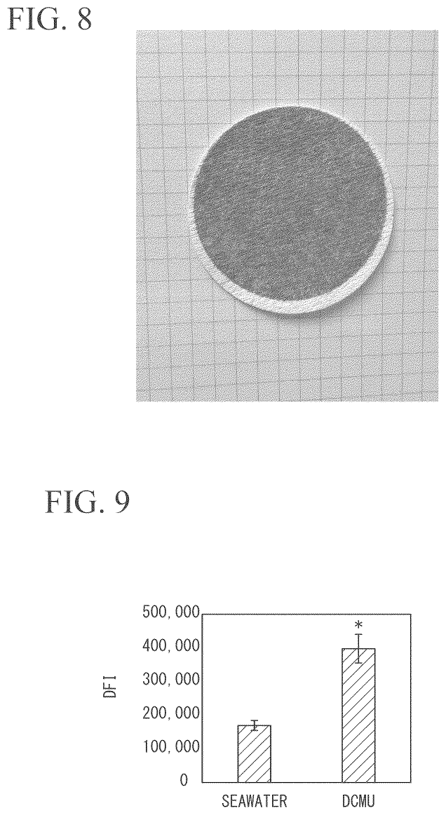

25 to 50 mL of a culture solution of NIES-981 was passed through a filter (Whatman GF/F glass fiber filter paper (diameter: 47 mm)) set in a filtration unit, capturing the cells on the filter. After the filtration, 1 mL of a protective medium (Table 6) was immediately added dropwise onto the filter. The filter on which the cells were captured was subjected to low-temperature vacuum drying for 1 to 2 hours using a low-temperature vacuum drying device (manufactured by Forest Technology Systems Ltd., FLEXI-DRY) capable of performing L-drying. Subsequent steps were performed in the same manner as in the preparation of the L-dried sample in the vial described above, thereby obtaining L-dried matter of algae on the filter (FIG. 8).

### Detection of Presence of Chemical Substance (2)

The L-dried matter obtained on the filter was exposed to seawater or test water containing DCMU at a concentration of 1 μM, and the delayed luminescence was measured 5 minutes after the exposure.

The results are shown in FIG. 9. It was found that there was a significant difference in the amount of delayed luminescence between the case of adding seawater and the case of adding DCMU (*P<0.01, N=4).

From the above results, it was demonstrated that, even in a case of using the dried algae on the filter, the assay for the target chemical substance could be performed very rapidly and with high accuracy by measuring the amount of delayed luminescence.

### Detection of Presence of Chemical Substance (3)

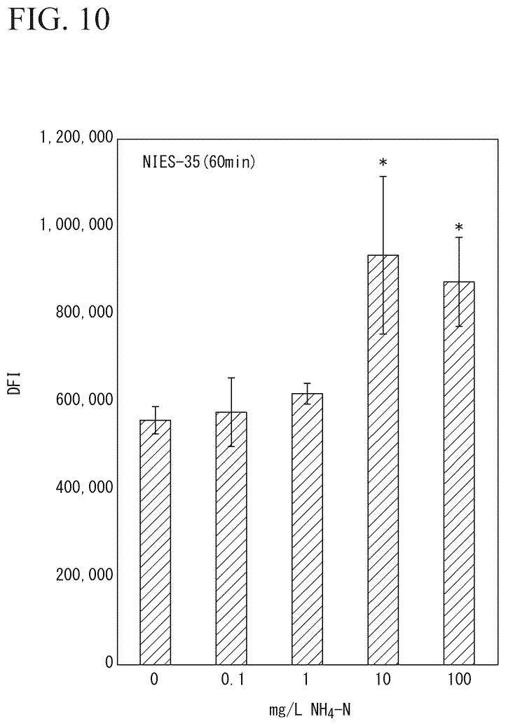

To the L-dried matter of NIES-35 obtained in the cryo vial, a sample containing ammonia nitrogen at a concentration shown in FIG. 10 was added, and the delayed luminescence was measured 60 minutes after the addition.

The results are shown in FIG. 10. In the case of adding NH₄—N at a concentration of 10 mg/L or more, a significant difference in the amount of delayed luminescence was confirmed (T-test, P<0.05, N=3).

The ammonia nitrogen is one of the most common chemical substances discharged from steel mills and agricultural drainage. For example, in coking waste liquid, ammonia nitrogen ranging from several hundred mg/L to several thousand mg/L is released. It is known that high concentrations of ammonia nitrogen are harmful to many organisms. In a case where a drainage standard for the ammonia nitrogen was set to 250 mg/L, it was possible to detect ammonia nitrogen at a concentration of 1/25 of that standard in the present assay.

### Estimation of Half-Effective Concentration

A dose-response curve of the L-dried matter with respect to a representative metal was created, and a half-effective concentration (EC₅₀) was estimated. For the L-dried matter of NIES-981, zinc at 0 to 1 ppm (w/v) or Cy metal mixed solution at 0 to 10% (w/v) was exposed simultaneously with rehydration. After 15 minutes, the delayed luminescence was measured, and a dose-response curve was created.

Since zinc is known as a metal species which is easily eluted from ores in hydrothermal regions, zinc was adopted as the representative metal here.

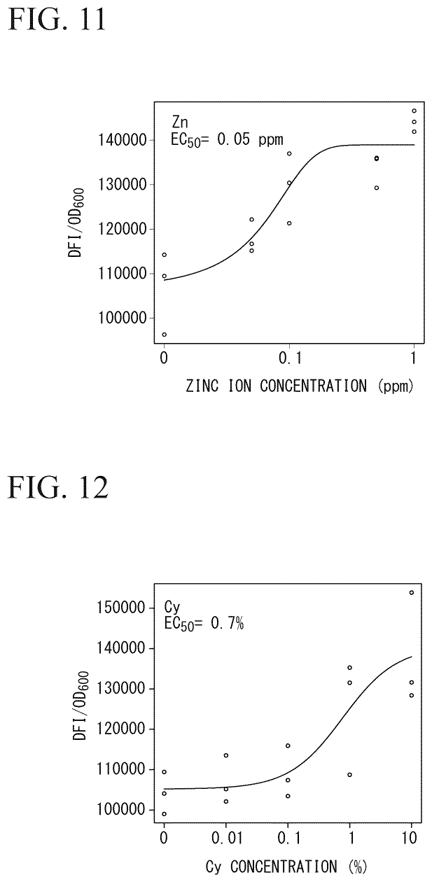

In a case where the half-effective concentration (EC₅₀) was calculated, the zinc was 0.05 ppm (w/v) (FIG. 11) and the Cy metal mixed solution was 0.7% (w/v) (FIG. 12). Although it is not possible to simply compare the values because the values were obtained by different bioassay methods, the EC₅₀ value during the zinc exposure was 0.07 ppm in a case where the 24-hour exposure test was performed on the same type of biocyanobacteria, and the EC₅₀ value was similar to this value.

In addition, in a case where the L-dried matter of NIES-35 was exposed to DCMU or HA for 15 minutes and a significant difference test was performed, DCMU was detectable even at 0.1 μM (approximately 0.02 ppm), and HA was detectable even at 0.0005 μM, which is an extremely low concentration. The concentration of HA corresponds to a 100 million-fold dilution of a commercially available stock solution.

In the measurement system of delayed luminescence using the L-dried matter, an extremely sensitive reaction was observed for chemical substances which directly affected the electron transfer system, such as DCMU and HA.

From these findings, it was demonstrated that, in the method adopted in the present invention, despite being able to be executed much more rapidly than the methods in the related art, the detection sensitivity was equal to or superior to that of the methods in the related art.

### Examination of Storage Conditions of L-Dried Matter

As described above, after the L-dried matter was produced, the DFI value was acquired in the same manner as the acquisition of the control for the detection of the presence of the chemical substance described above, in which the L-dried matter was stored at 4° C. or 37° C. under atmospheric pressure unlike the L-drying method in the related art, or the L-dried matter was dried in a glass ampule, and an opening of the ampule was sealed with a molten seal while maintaining a vacuum according to the L-drying method in the related art.

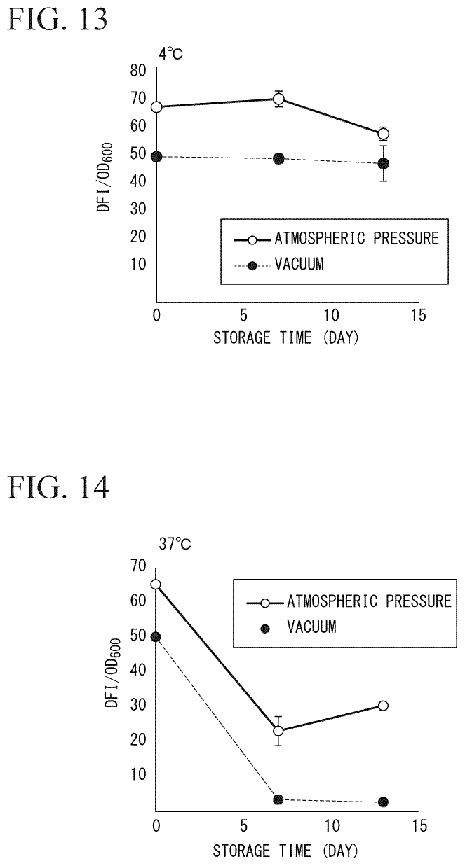

The results are shown in FIGS. 13 and 14. Surprisingly, a higher DFI value was measured when the storage was performed under atmospheric pressure, compared to when the storage was performed under vacuum. From the above, it was found that omitting the enclosing operation for vacuum storage did not affect the quality of the L-dried matter, and in fact improved the quality.

By omitting the enclosing operation for vacuum storage, not only was the work process simplified and the production efficiency of the L-dried matter significantly improved, but it was also possible to produce a high-quality L-dried matter.

### Accelerated Degradation Test of L-Dried Matter

The L-dried matter of NIES-981 obtained above was stored at 37° C. for 1 to 10 days under atmospheric pressure. The detailed changes in the DFI value were acquired in the same manner as the acquisition of the control for the detection of the presence of the chemical substance described above.

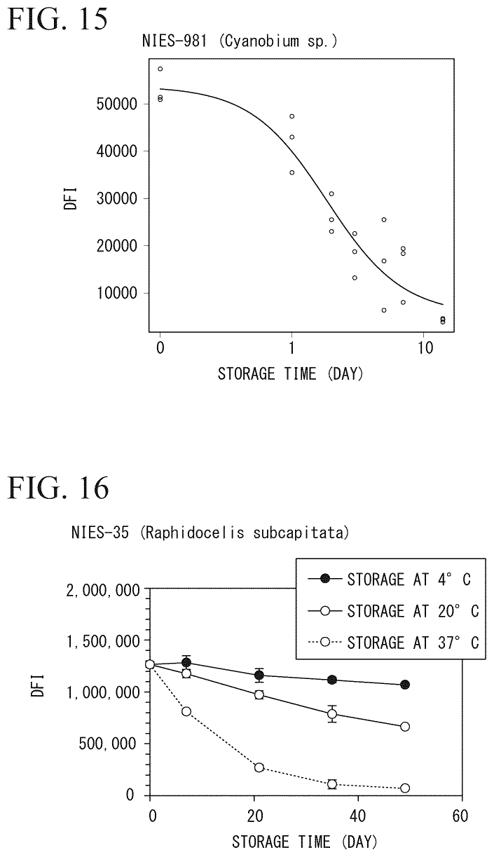

The results are shown in FIG. 15. It was found that the L-dried matter gradually deteriorated based on the decrease in the DFI value over the storage period. However, assuming that storage at 37° C. for 10 days was equivalent to storage at 4° C. for 20 years, the estimated half-life (time at which the delayed luminescence was reduced to half) for NIES-981 was calculated to be approximately 2.6 years, it was estimated that long-term storage at low temperatures was feasible.

In addition, the L-dried matter of NIES-35 or NIES-981 was stored at 4° C., 20° C., or 37° C. under atmospheric pressure, and the DFI value was measured in the same manner.

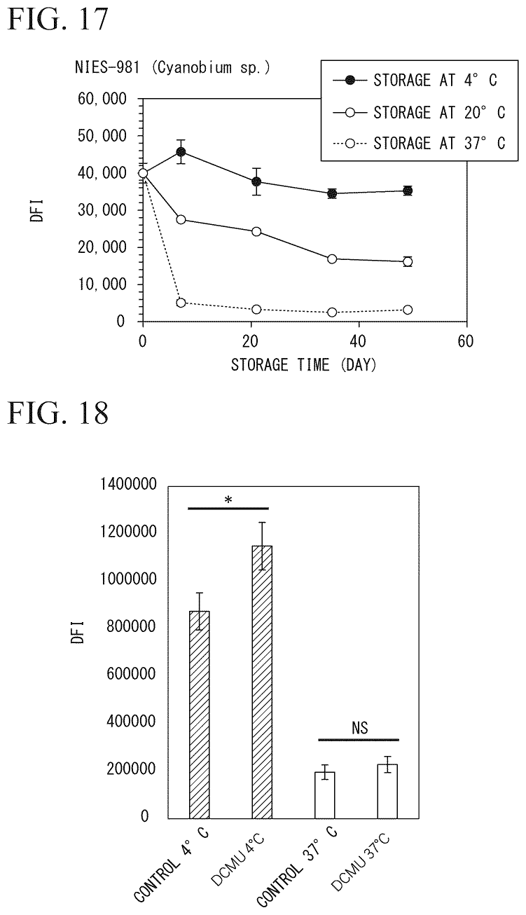

The results are shown in FIGS. 16 and 17. As compared to the storage at 37° C., the deterioration of the DFI was suppressed under storage conditions at 20° C., and it was expected that long-term storage was possible even at the easily maintainable temperature of 20° C.

### Quality Evaluation of L-Dried Matter

The L-dried matter stored at 4° C. for 2 weeks was compared with the L-dried matter stored at 37° C. for 2 weeks (accelerated deterioration product). In each case, DCMU was added to the L-dried matter of NIES-35 at the same time as the rehydration, such that the final concentration was 1 μM (DCMU addition group), and the delayed luminescence after 15 minutes was measured. In addition, the delayed luminescence was measured in the control (non-addition group) in which DCMU was not added, following the same procedure.

In the L-dried matter stored at 4° C., a difference was observed between the DCMU-added group and the non-addition group. However, in the L-dried matter stored at 37° C., no significant difference was observed between the DCMU-added group and the addition group, confirming the deterioration of the L-dried matter stored at 37° C. (FIG. 18).

As described above, by using electron transfer inhibitors such as DCMU, it was possible to determine the quality of the L-dried matter in a very short time.

The features described in the respective embodiments such as configurations and combinations of the configurations are only illustrative. Therefore, it is possible to add other configurations or to omit, replace or modify the configurations described herein without departing from the spirit of the present invention. Further, the scope of the invention is not limited to the embodiments described hereinabove and is limited only by the Claims.

### REFERENCE SIGNS LIST

10: Dried algae
20, 21: Sample
22: Test substance
30: Water
40, 41, 42: Measurement object

1. A detection method for detecting a presence or absence of a test substance in a sample, the detection method comprising:
measuring a delayed luminescence A of a measurement object by bringing an L-dried matter of algae, which is dried algae obtained by an L-drying method, into contact with the sample,
wherein, in the measuring, the delayed luminescence A of the measurement object is measured by bringing the dried algae into contact with water and the sample, or by bringing the dried algae into contact with the sample containing water, and
in the measuring, a value of the delayed luminescence A is measured within 90 minutes from a point of time at which the dried algae are brought into contact with the water.

2. A detection method for detecting a presence or absence of a test substance in a sample, the detection method comprising:
measuring a delayed luminescence A of a measurement object obtained by directly bringing dried algae into contact with the sample containing water,
wherein, in the measuring, a value of the delayed luminescence A is measured within 90 minutes from a point of time at which the dried algae are brought into contact with the water.

3. (canceled)

4. The detection method according to claim 1,
wherein the presence or absence of the test substance in the sample is determined based on a comparison result between a value of a delayed luminescence B of a control measurement object and a value of the delayed luminescence A.

5. The detection method according to claim 1,
wherein the test substance contains an electron transfer inhibitor which inhibits an electron transfer reaction in a photosynthesis process.

6. The detection method according to claim 1,
wherein the test substance contains a heavy metal.

7. The detection method according to claim 1, further comprising:
a drying step of drying algae to obtain the dried algae.

8. The detection method according to claim 7,
wherein a drying method is an L-drying method.

9. The detection method according to claim 1,
wherein the dried algae are on a filter.

10. The detection method according to claim 1,
wherein algae of the dried algae include one or more kinds of algae selected from the group consisting of algae belonging to Raphidocelis genus, algae belonging to Synechococcus genus, algae belonging to Cyanobium genus, algae belonging to Prochlorococcus genus, algae belonging to Chlorella genus, and algae belonging to Parachlorella genus.

11. The detection method according to claim 1,
wherein algae of the dried algae include one or more kinds of algae selected from the group consisting of Raphidocelis subcapitata NIES-35, Synechococcus sp. NIES-969, Cyanobium sp. NIES-981, and Prochlorococcus sp. NIES-2885.

12-24. (canceled)

25. A dried algae quality management method which is a quality management method of the dried algae used in the detection method according to claim 1, the quality management method comprising:
bringing the dried algae into contact with an electron transfer inhibitor which inhibits electron transfer in a photosynthesis process to measure a delayed luminescence A′; and
evaluating quality of the dried algae based on a comparison result between a value of a delayed luminescence B′ of a control measurement object and a value of the delayed luminescence A′.

26. The quality management method according to claim 25,
wherein the electron transfer inhibitor is one or more selected from the group consisting of 3-(3,4-dichlorophenyl)-1,1-dimethylurea (DCMU), hydroxylamine (HA), and sodium azide.

27. A dried algae quality management method which is a quality management method of dried algae used in a detection method for detecting a presence or absence of a test substance in a sample, the detection method including measuring a delayed luminescence A of a measurement object obtained by bringing dried algae into contact with the sample, the quality management method comprising:
bringing the dried algae into contact with an electron transfer inhibitor which inhibits electron transfer in a photosynthesis process to measure a delayed luminescence A′; and
evaluating quality of the dried algae based on a comparison result between a value of a delayed luminescence B′ of a control measurement object and a value of the delayed luminescence A′.

28. The quality management method according to claim 27,
wherein the electron transfer inhibitor is one or more selected from the group consisting of 3-(3,4-dichlorophenyl)-1,1-dimethylurea (DCMU), hydroxylamine (HA), and sodium azide.
